(19) United States
(12) Patent Application Publication
(10) Pub. No.: US 2004/0220086 A1
(43) Pub. Date: Nov. 4, 2004

(54) Methods and materials for targeting and affecting selected cells

(76) Inventor: W. Page Faulk, Indianapolis, IN (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/493,033

(22) PCT Filed: Oct. 17, 2002
(86) PCT No.: PCT/US02/31582

(30) Foreign Application Priority Data
Oct. 17, 2001 (US) 60329539

(51) Int. Cl. A61K 38/16

(52) U.S. Cl. 514/6

(57) ABSTRACT

Diseased tissue is treated by contact with a protein that preferentially binds to cells in the diseased tissue, the protein carrying with it a plurality of cell-affecting entities.

### FIELD OF THE INVENTION

This invention relates generally to methods and materials for targeting and affecting selected cells in a living organism and more specifically to preferentially delivering cell-affecting materials to cells having a relatively high incidence of transferrin receptors for treating or for imaging such cells, or both.

### BACKGROUND

Two of the most devastating and intractable problems in cancer treatment are drug-toxicity, which debilitates patients, and drug-resistance, which requires more drugs and thus amplifies the problem of drug-toxicity, often resulting in death. One solution that is being evaluated to solve the problem of drug-toxicity is to deliver drugs primarily to targeted cells, such as cancer cells. Many researchers are working to develop antibodies against cancer cells that will carry anticancer drugs to their target. This approach holds promise, but antibodies are not without problems. For example, they often cross-react with normal tissues, and they can damage blood vessels (e.g., vascular leak syndrome), and cause dangerous allergic reactions (e.g. anaphylaxis).

Drug targeting spares normal cells, requires less drug, and significantly diminishes drug-toxicity. When anticancer drugs are not delivered selectively to diseased cells, their toxicities particularly damage the immune system and the system responsible for blood clotting. Thus, infections and bleeding are principal complications of chemotherapy in cancer patients. These complications require expensive and often uncomfortable services, treatments, hospitalizations, intensive care, and life-support systems. Such problems are largely preventable by targeted drug delivery.

The problem of drug-toxicity consumes huge blocks of time from doctors and nurses, and is responsible for much of the cost of cancer care. For example, it is commonly accepted that 70% of calls to oncologists are due to a problem of drug-toxicity. Today there is no satisfactory way to treat drug-toxicity except to use less drug. Targeted delivery allows the use of less drug, because substantially all of the administered drug is delivered specifically to the target on cancer cells rather than being nonspecifically distributed around the body. This solution to the problem of drug-toxicity will dramatically transform the treatment of cancer patients.

The problem of drug-resistance is equally serious. Typically this problem occurs when a cancer patient is treated and responds with a symptomless remission that lasts many months but that is followed by a return of the cancer in a form that no longer responds to any known drug. Today there is no satisfactory solution, except the use of larger amounts of more powerful drugs, which causes serious drug-toxicity problems, often resulting in the death of the patient. However, targeted drug delivery can overcome the problem of drug-resistance.

The targeting of cancer cells by non-antibody proteins has shown promise in the recent past by the use of tumor affecting agents linked to transferrin. Background research for the use of transferrin to target cells began in the 1970s with studies of extra-embryonic tissues for onco-fetal antigens. This revealed transferrin receptors on extra-embryonic trophoblast (1-4), but not on extra-embryonic amniotic epithelium (5). However, when amniotic epithelial cells were grown in culture they produced transferrin receptors (6). The receptors then were identified on different types of cultured cells (7), while they were absent from normal (i.e., uncultured) cells. These findings prompted Faulk and colleagues to study cancer biopsies, which led to the original 1980 report of transferrin receptors on breast adenocarcinoma cells (8). This was followed by a 1984 report of transferrin receptors on the surface of lymphoma, myeloma and leukemia cells (9). These findings have been confirmed and extended many times (for review, see reference 10). Human cancers in which transferrin receptors have been identified are listed in the following Table.

| Tumor Studied | References |
|---|---|
| Breast | 8, 11 |
| Leukemia | 44, 12 |
| Lung | 13 |
| Brain | 14 |
| Liver | 15 |
| Bladder | 16 |
| Gastrointestinal | 17 |
| Ovary | 18 |
| Non-Hodgkin's lymphoma | 19 |
| Lymphoma/melanoma | 9, 20 |
| Nasopharyngeal | 150 |
| Cervix | 151 |

Background: Transferrin Receptors on Normal and Cancer Cells.

No single study has asked if all human cancers have up-regulated transferrin receptors, or if all normal cells have down-regulated transferrin receptors, but data from many quarters suggest that the answer to both questions is a qualified yes. Immature erythrocytes (i.e., normoblasts and reticulocytes) have transferrin receptors on their surfaces, but mature erythrocytes do not (21). Circulating monocytes also do not have up-regulated transferrin receptors (22), and macrophages, including Kupffer cells, acquire most of their iron by a transferrin-independent method of erythrophagocytosis (23). In fact, virtually no iron enters the reticuloendothelial system from plasma transferrin (for review, see reference 24). Macrophage transferrin receptors are down-regulated by cytokines such as gamma interferon (25), presumably as a mechanism of iron-restriction to kill intracellular parasites (26).

In resting lymphocytes, the gene for transferrin receptor is not even measurable (27), but stimulated lymphocytes up-regulate transferrin receptors in late G₁ (28). Receptor expression occurs subsequent to expression of the c-myc proto-oncogene and following up-regulation of IL-2 receptor (29), and is accompanied by a measurable increase in iron-regulatory protein binding activity (30), which stabilizes transferrin receptor mRNA (31). This is true for both T and B lymphocytes (32), and is an IL-2-dependent response (33).

Up-and-down regulation of transferrin receptors for normal and tumor cells has been shown by studies of antigen or lectin stimulation (i.e., receptor up-regulation), and by studies of differentiation models (34-37) using retinoic acid (i.e., receptor down-regulation). Base-line data from these experimental models suggest that these receptors are down-regulated from the plasma membranes of most normal, adult, resting human cells (38). Exceptions are the circulatory barrier systems, which include the materno-fetal barrier with its transferrin receptor-rich syncytiotrophoblast (39); the blood-brain barrier with its transferrin receptor-rich capillary endothelial cells (40); and, the blood-testis barrier with its transferrin receptor-rich Sertoli cells (41).

Little is known about the molecular biology of these specialized tissues, but it is known that they do not traffic intracellular iron in the same way as other tissues. For example, after binding to transferrin receptors on Sertoli cells, the transferrin-iron complex is internalized as by other cells, but the iron then is transferred to another transferrin produced by Sertoli cells, and transported to transferrin receptors on spermatocytes (42). It is not known if these normally up-regulated transferrin receptors will contribute to toxicity of transferrin-drug conjugates, or if they will offer privileged access in the treatment of testicular, trophoblastic or brain cancers.

Background: Transferrin-Drug Conjugates.

The concept of using transferrin to deliver anticancer drugs was proposed in 1980 (8). A method for the preparation of transferrin-doxorubicin conjugates was published in 1984, which presented data on the sensitivity and specificity for killing human HL60 and Daudi cells (43), as well as for killing peripheral blood and bone marrow mononuclear cells from leukemia patients (44). These reports prompted other reports of methods for the preparation of transferrin-drug conjugates, some of which are listed in the following Table.

| Transferrin Label | Method Used | Refs |
|---|---|---|
| Doxorubicin | Glutaraldehyde | 43, 45, 46 |
| Doxorubicin | Maleimide | 47 |
| Mitomycin C | Glutaryl Spacer | 48 |
| Neocarzinostatin | Succinimide | 49 |
| Diphtheria Toxin | Thioester | 50 |
| Chlorambucil | Maleimide | 51 |
| Paclitaxol | Glutaraldehyde | 52 |
| Daunorubicin | Glutaraldehyde | 53 |
| Titanium | Carbonate | 54 |
| Insulin | Disulfide | 55 |
| Gallium | Carbonate | 56 |
| Platinum | Methionine | 57 |
| Saporin/ricin | Succinimide | 58 |
| Ruthenium | Bicarbonate | 59 |
| Growth Factor | Fusion Protein | 60 |
| HIV Protease | Recombinant | 61 |

Transferrin conjugates of doxorubicin can be prepared by glutaraldehyde-mediated Schiff base formation (62, 63), which forms an acid-resistant bond between epsilon-amino lysine groups of transferrin and the 3′amino position of doxorubicin. However, if doxorubicin is conjugated to antibodies through an acid-sensitive bond, such as that formed by using a hydrazone linker, the targeted doxorubicin is more cytotoxic (64,65). Observations such as these led to an idea that drugs bound to carriers by acid-sensitive bonds release drugs within cells and thus are more effective than drugs bound to their carriers by acid-resistant bonds (64-66). This idea is compatible with the DNA-intercalation mechanism of doxorubicin cytotoxicity (67), but it is not compatible with the plasma membrane-mediated mechanisms of doxorubicin cytotoxicity (for review, see reference 68).

Although DNA intercalation is an established mechanism of cell death by doxorubicin, immobilized doxorubicin on carriers, such as dextran, activate plasma membrane-mediated mechanisms to kill cells (69,70). In this regard, there are several striking biochemical analogies between immobilized doxorubicin and glutaraldehyde-prepared transferrin-doxorubicin conjugates. First, they both are acid-resistant (71); second, they both initiate plasma membrane and signal transduction reactions (72); third, they both are endocytosed very slowly (73); and fourth, they both fail to transport doxorubicin into the nucleus (74). It thus appears that conjugates of doxorubicin with transferrin kill cells by activating plasma membrane-mediated mechanisms that involve both doxorubicin and transferrin receptors. These mechanisms are discussed in the following section.

Background: Mechanisms of Cell Killing by Transferrin-Drug Conjugates.

Transferrin-doxorubicin conjugates bind to plasma membranes by sequentially employing two mechanisms; initially the transferrin component is bound by transferrin receptors, after which the doxorubicin component is bound by the lipid bilayer, primarily by interacting with cardiolipin and charged phosphates (68). The sequence of these events is supported by observations that conjugates do not bind to either normal or transferrin receptor-negative cells (45), and that substantially more transferrin is required to displace transferrin-doxorubicin than transferrin from receptor-positive cells (75,76). Thus, bound through protein and phospholipid receptors, the conjugates are positioned to activate signal transduction pathways by receptor dimerization, lateral mobility and cytoplasmic calcium mobilization (77).

The most studied pathway activated by ligand-receptor interaction for transferrin is endocytosis (for review, see references 78 and 79), but several other pathways are activated that are important in the selective killing of cancer cells by transferrin-doxorubicin conjugates. Foremost among these is NADH-oxidase, a major redox enzyme located in plasma membranes (80). This enzyme is activated (81) when transferrin receptor binds its ligand (i.e., transferrin). Inhibition of NADH-oxidase causes cell death (82), and doxorubicin is an efficient inhibitor of this enzyme (83,84). Transferrin-doxorubicin conjugates inhibit NADH-oxidase (85), as well as down-stream reactions initiated by NADH oxidation, such as loss of electrons and exchange of protons through the sodium-hydrogen antiport (72). Thus, inhibition of plasma membrane redox enzymes, particularly NADH-oxidase, is one mechanism involved in the killing of tumor cells by transferrin-doxorubicin conjugates (86).

Another mechanism of cell killing by transferrin-doxorubicin conjugates involves the molecular control of transferrin receptors. This is illustrated by the markedly different responses of normal and cancer cells to restricted microenvironmental iron. For example, chelation of microenviromental iron initiates apoptosis in tumor cells but not in normal resting cells (87), and such chelation enhances significantly the cytotoxic effect of cytosine arabinoside (88). Drug-resistant cells are much more sensitive to iron restriction, due to their inability to stabilize transferrin receptor mRNA (unpublished results), and excess iron destabilizes transferrin receptor mRNA more effectively in drug-resistant than in drug-sensitive cells (89). Additional studies of this molecular model of drug-resistance have revealed that sodium nitroprusside, which nitrosylates the iron-sulfur cluster of the iron-regulatory protein, mediates destabilization of transferrin receptor mRNA, and that drug-resistant cells are significantly more susceptible than drug-sensitive cells to this iron-independent mechanism (89).

Another group of iron-independent switches controlling the molecular machinery of post-translational regulation of transferrin receptors are redox-active products of oxidative stress (for review, see reference 90). For example, nitric oxide disassembles the iron-sulfur cluster, allowing iron-regulatory proteins to bind and protect iron-response elements (91), and the kinetics of this reaction closely resemble iron-mediated control of iron-sulfur clusters in iron-regulatory proteins (92). Also, hydrogen peroxide causes the same effect (i.e., up-regulation of transferrin receptors), but the hydrogen peroxide reaction is significantly more rapid than that initiated by nitric oxide (93). Similarly, transferrin receptors are down-regulated by the nitrosium ion, which causes nitrosylation of thiol groups within the iron-sulfur cluster (94). Thus, investigations of iron-dependent pathways may not reveal why transferrin receptors are up-regulated in human cancer. Certainly, iron-independent pathways activated by cytokines (95,96), free radicals (90,93) and nitrosylation (97) affect both receptor regulation and cytotoxicity.

Background: Transferrin-Drug Conjugates in Laboratory Animals.

The efficacy of transferrin-drug conjugates has been investigated in several animal models. For example, the ability of transferrin-diphtheria toxin conjugates to kill human glioma cells in nude mice has been studied and found to decrease the gliomas by 95% on day 14, and the gliomas did not recur by day 30 (98). Another study investigated the efficacy of glutaraldehyde-prepared transferrin-doxorubicin conjugates to rescue nude mice from death by human mesothelioma cells, and found that the conjugates significantly prolonged life compared to animals treated only with doxorubicin (99). There also are reports of targeting cytolytic viruses as conjugates of transferrin to tumor cells. For example, transferrin has been conjugated to herpes simplex virus thymidine kinase by using biotin-streptavidin technology, and these conjugates have prolonged life in immune-deficient mice inoculated with metastasizing K562 tumor cells (100).

There have been no comprehensive studies of the toxicity or pharmacokinetics of transferrin-drug conjugates, although there are data that human transferrin binds to mouse, rat, monkey and human transferrin receptors with similar affinity (101). In light of this, the toxicity of human transferrin-chlorambucil conjugates studied in mice was found to be less toxic than free chlorambucil, for mice receiving free drug died and mice receiving conjugates survived (51). Similarly, the maximum tolerated dose of doxorubicin in human transferrin-doxorubicin conjugates in nude mice was found to be 20 mg/kg (iv) for conjugates and only 8 mg/kg (iv) for free drug (47). Studies of human transferrin-neocarzinostatin in nude mice revealed a half-life of 55 minutes, while that for free neocarzinostatin was 7 minutes, and the conjugates produced no ill effects on either liver or kidney function (102).

Transferrin-Drug Conjugates in Human Patients.

There are only two clinical reports of transferrin-drug conjugates in human cancer patients. The first paper was published in 1990, which was a preliminary study of seven acute leukemia patients treated intravenously with 1 mg/day of glutaraldehyde-prepared transferrin-doxorubicin conjugates for 5 days. With these low doses, there were no toxic effects, and the number of leukemic cells in peripheral blood of the 7 patients decreased by 86% within 10-days following therapy (103). In addition, there was no extension of disease as assessed by examination of bone marrow biopsies before and after treatment (103). The same transferrin-doxorubicin conjugates have been shown to kill selectively leukemic cells from peripheral blood and marrow of leukemia patients (44).

The second clinical report was published from the National Institute of Neurological Diseases and Stroke in 1997, and involved 15 patients with recurrent brain cancers treated with thioether-bonded transferrin conjugates of a genetic mutant of diphtheria toxin (50). The conjugates were delivered by high-flow interstitial microinfusion, which has been shown to produce effective perfusion of radiolabeled transferrin in primate brains with minimal inflammatory responses (104). Magnetic resonance imaging revealed at least a 50% reduction in tumor volume in 9 of the 15 patients, including 2 cases of complete remission (50).

Though presently unpublished, there is another clinical study of 23 patients with advanced ovarian cancer who were randomized into test (12 patients) and placebo (11 patients) groups. The test group received transferrin-doxorubicin conjugates equivalent to 1 mg doxorubicin per day on days 15 through 19 of monthly treatment cycles. A significant difference was revealed by Cox regression estimates of survival rates for patients treated with transferrin-doxorubicin conjugates when the time between diagnosis and randomization was 18 months (manuscript in preparation).

The only other clinical investigation of transferrin-doxorubicin conjugates also is not yet published. This concerns a 22-year old male with metastatic disease from a sarcoma of his right atrium who was treated beginning August, 2000 by using conventional protocols. The patient failed conventional chemotherapy and by November, 2000, he was suffering from drug toxicities, his lungs were filled with metastatic lesions, and he was coughing blood-stained sputum when his physician father obtained an IND from the FDA for the use of transferrin-doxorubicin conjugates, and treatment was begun in November, 2000 By January, 2000, the patient's lungs were substantially cleared of metastatic lesions, and there was no radiological evidence of tumor. He presently (August 2001) is active, receiving only transferrin-doxorubicin. (Case report in preparation).

The targeted delivery of drugs has a remarkable advantage of delivering less drug to patients, thereby increasing efficacy, decreasing costs and minimizing toxicity by causing less collateral damage to normal cells. Targeted delivery addresses the central problem of drug toxicity, but another central problem in the treatment of cancer is drug-resistance. Although there are several mechanisms of drug-resistance (e.g., efflux pumps), they share a common characteristic of being activated by the non-specific entrance of drugs into cells (105). In this regard, transferrin is a particularly interesting carrier, because it enters cells by employing a receptor-specific pathway (78). Thus, transferrin-drug conjugates might be trafficked around drug-resistance mechanisms such as efflux pumps in resistant cells (85).

Data published in 1992 indicated that transferrin-doxorubicin conjugates were effective in killing K562 and HL60 cells that were resistant to doxorubicin (106). These findings were confirmed independently in 1993 with drug-resistant K562 cells (107), and were reconfirmed and extended to other types of drug-resistant cells in 1994 (108), 1996 (109), and 2000 (110). Interestingly, doxorubicin immobilized on solid carriers such as dextran (70) or nanoparticles (111) also have been shown to be effective against doxorubicin-resistant cells. In fact, a concept is emerging that vectorization of doxorubicin with one of several peptide vectors is effective in overcoming multidrug resistance (112). In summary, both vectorized/immobilized doxorubicin and transferrin-doxorubicin conjugates kill drug-resistant cancer cells (68,69,106,109,112) by activating plasma membrane-mediated reactions that activate signal transduction pathways, which result in cell death.

### SUMMARY OF THE INVENTION

Although targeted delivery of cell-affecting materials such as doxorubicin by transferrin-doxorubicin conjugates avoids many of the problems of the prior art, improvements in the efficiency and effectiveness of treatments for drug-resistant cancer cells are always welcome. Such improvements are provided by the present invention. In one aspect, the present invention comprises proteins that are selectively attracted to certain cells, such as cancer cells, the proteins being adapted to carry with them a plurality of different cell-affecting entities. The preferred protein at the moment is transferrin because it is attracted in relatively high concentrations to cancer cells, although other proteins that are attracted to receptors found in relatively high numbers on selected cells may also be used. The cell-affecting entities preferably affect the targeted cell with different mechanisms of action. For example, one cell-affecting entity carried by the protein may be a drug, such as doxorubicin, while a second cell-affecting entity may be a radioisotope of a metal such as Bismuth or may be a non-radioactive metal known to have a desired affect on the targeted cells. The second cell-affecting entity may be a material such as gallium that is also useful in imaging the targeted cells. Further, the conjugate may be adapted to carry more than two cell-affecting entities in a wide variety of combinations.

In another aspect the invention comprises a method of making such proteins.

In still another aspect the invention comprises a method for treating diseases by selective application of proteins carrying a plurality of different cell effecting entities.

### DETAILED DESCRIPTION

Synthesis of the Conjugates:

Synthesis of metal-loaded transferrin-doxorubicin conjugates was accomplished by first preparing metal-free transferrin-doxorubicin conjugates, although it will be readily understood by those familiar with the manipulation of proteins that transferrin-metal conjugates could be prepared first. The original method used a mixture of transferrin and doxorubicin with the bivalent linker glutaraldehyde (43), but this produced dimers and aggregates. This method subsequently was improved so that aggregates were not formed (45), but chromatography used in this method diminished the yield of homogenous conjugates. It is preferred to employ glutaraldehyde as a linker to produce high yields of homogenous conjugates containing a defined and consistent number of molecules of doxorubicin per molecule of transferrin without using chromatography. The transferrin (99% purity) can be purchased from Kamada, Ltd. (Rehovot, Israel), and the doxorubicin can be purchased from Ben Venue, Inc. (Bedford, Ohio). The preferred method of making conjugates is disclosed in International Application PCT/US02/11891 of the present inventor, the disclosure of which is hereby incorporated by reference.

Following preparation of the metal-free transferrin-doxorubicin conjugates, the metal-binding sites of transferrin were loaded with metals that are known to have stable binding constants for the two metal-binding sites situated in the interdomain clefts of the N-lobe and C-lobe of transferrin (113). As mentioned above, the loading of the metals could have occurred prior to adding or linking a drug, such as doxorubicin, to the protein. Further, it is within the scope of the invention to load the protein with a plurality of cell-effecting entities that do not include a drug. For example, the cell-effecting entities could be one or more cancer killing metals, cancer killing isotopes, imaging entities or various combinations such entities.

Metal loading of the transferrin molecules is not a safety issue for patients. There is a redundant capacity for metal binding by transferrin because only 30% of the transferrin molecules in plasma normally are occupied in carrying iron (115). For iron, this generally is known as the iron-binding capacity (41). There is no free iron in plasma (24), so metals of lower binding affinities will not be displaced from transferrin in vivo. Also, although albumin can bind certain metals, its binding affinity is less than that of transferrin (123), so there is no danger of losing the metals from transferrin to albumin in vivo.

There are 30 metals known to be transported by transferrin (114). Thermodynamic data indicate that very few of these have stability constants (i.e., log K values) above 6 to 8 (e.g., log K values for nickel and zinc are 4.1 and 7.8, respectively), while iron has a log K value of about 20 (115). Research to define which of the 30 metals have physical-chemical properties that allow them to be loaded into the metal-binding sites of transferrin has revealed that gallium, bismuth, aluminum and ruthenium have appropriate ionic radii to fit the interdomain clefts. Also, upon being loaded into the metal-binding sites, these metals generate conformational shifts that allow the molecule to be bound by transferrin receptors. References for four such metals are given in the following Table.

Physico-Mechanistic Properties of Selected Metals

| | | | Causes | |
|---|---|---|---|---|
| | Ionic | Stability | Conformational | |
| Selected | Radius | Constants | Shift in | Mechanism of |
| Metals | (Å) | (log K₁*) | Transferrin | Cell Killing |
| Gallium | 0.62 | 19.5 (115) | (117, 118) | Activates |
| | | | | lysosomes (121) |
| Aluminum | 0.54 | 15.4 (115) | (119) | Lipid |
| | | | | peroxidation (122) |
| Bismuth | 0.96 | 19.4 (116) | (120) | Thiolate |
| | | | | binding (123) |
| Ruthenium | 0.67 | unknown | (138) | DNA damage (124) |

Although albumin has a major role in the intravascular transport of many metals, transferrin appears to be the principal transporter of gallium, aluminum, bismuth and ruthenium. The cytotoxic properties of these metals (132-138) also can be utilized, because the conformational changes they induce in transferrin are spatially appropriate to allow the transferrin-metal complexes to be recognized and bound by transferrin receptors. These biomedical properties have prompted limited clinical studies of selected transferrin-metal complexes as targeted therapeutic tools in cancer patients. References for published papers supporting these statements are listed in the following Table.

Properties of Selected Metals and Their Complexes with Transferrin

| | Selective | Complex Fits | | | |
|---|---|---|---|---|---|
| | Transferrin | Transferrin | Cytotoxic | Cytotoxic | |
| Selected Metals | Binding | Receptor | In Vitro | In Vivo | Clinical Studies |
| Gallium | (125) | (130, 149) | (132) | (139) | (143) |
| Aluminum | (126-128) | (131) | (133-135) | (140) | (140) |
| Bismuth | (116, 123) | (54) | (158) | (159, 160) | (144, 157) |
| Ruthenium | (129) | (156) | (136-138) | (141, 142) | (145, 146) |

The metals were loaded separately into the two binding sites of transferrin by pH-dependent reactions that involve presentation of the metals weakly chelated with citrate in the presence of bicarbonate at an acidic ph (e.g., 4.9), and the pH is slowly increased to physiological conditions over several hours (e.g., 3). This method assures an opened cleft for binding at acidic pH and a closed cleft for stability at physiological pH. Within the cleft, each inserted metal is nested by the phenolate oxygens of two tyrosine residues, an imidazole nitrogen of a histidine residue, a carboxylate oxygen of an aspartic acid residue, and two oxygens of the synergistic bicarbonate anion (147). Conjugates in solution were found to be stable and active for 6-9 months, and lyophilized conjugates were found to be stable and active for at least one year. It will be readily apparent to those of ordinary skill in the art that the loading of other metals into protein binding sights, or the linking of other metals to transferring or to other protein, may be accomplished at different pH values or with different procedures, all of which are intended to be within the scope of this invention.

Isotopes of the metals also can be used for their cell-affecting properties or for their imaging qualities, or both, and combinations of the metals or of metals and isotopes can be used. For example, a transferrin-doxorubicin conjugate can be loaded with a ruthenium atom and a bismuth isotope atom to take advantage of the cell-killing properties of the bismuth isotope and of the imaging qualities of the ruthenium isotope. Thus, the metals used in the present invention may be isotopic or nonisotopic or a combination thereof. When a drug is attached to a protein, such as transferrin, normally 0.5 to 2.5 molecules of the drug will be attached to one molecule of the protein. It is preferred that I to 2 molecules of the drug be present in the complex for every molecule of protein, and most preferably about 1.5 molecules of the drug are present per molecule of the protein.

When a metal, either isotopic or non-isotopic, is present in the protein, the amounts can vary depending upon the particular protein chosen and the manner of placing the metal on or in the protein. In the case of transferrin, there are two iron binding sites available, and one or two molecules of the metal will be present per mole of transferrin, although, as can be readily appreciated, mixtures of transferrin containing one atom of metal and transferrin molecules containing two atoms of metal can be used. The metals in the iron binding sites of transferrin can be the same or different. For instants, one of the binding sites can contain Bismuth for its anti-cancer effect, and the other iron binding site could contain Gallium for its imaging ability.

When an isotope or other metal is captured in one of the iron binding sites of transferrin, it will preferably be used for the treatment of tumors in the following amount, based on the amount of the isotope:

Gallium—67=5-15 mCi

Bismuth—213=0.2-0.6 mCi/kg with total doses of 10-45 mCI

Ruthenium 20-50 mg/kg/day

Cisplatin=75 mg/meter squared

Iron as the isotope iron—52=50-65 mCi

For the above isotopes, the isotope of Gallium is used for imaging, or diagnosis, whereas Bismuth and Iron are used as the isotope for treatment, and Ruthenium is used in the non-isotope form for treatment. The Cisplatin identified above is used in a non-isotopic form of platinum for treatment. The Cisplatin is bound to transferrin through an amino acid thought to be within the iron-binding site, which is a binding mechanism quite different from that for doxorubicin described above. It appears that the Cisplatin binds by a different mechanism than just slipping into the iron-binding site, like for instance Gallium does. There are data showing specific interactions of platinum with an amino acid that has electrons at the proper energy level. Thus, the binding of Cisplatin is believed to be a protein-metal binding, and as such it is due to the platinum.

The treatment and imaging conjugates of the present invention also includes chelator-bound transferrin-isotope conjugates. For the purposes of this specification, chelators are molecules that contain sufficient reactive sites to provide one that attaches to transferrin and another, which is a strong cation-binding site, that selectively binds certain isotopes. It is essential that these attachments are stable, for free isotope can depress the immune system and render patients susceptible to life-threatening infections.

Several chelators have been reported as bifunctional reagents which bind isotopes and protein carriers, such as antibodies. However, there are very few reports of chelators that bind transferrin as the targeting agent. In light of this, chelators were studied as bifunctional reagents for transferrin and different isotopes. This work has identified two chelators that yield stable transferrin-isotope conjugates. These molecules are diethylenetriaminepentaacetic acid (DTPA) and 1,4,7,10-tetraazacyclododecane-1,4,7, 10-tetraacetic acid (DOTA). Thus, both of these chelators have been used in the preparation of chelator-bound transferrin-isotope conjugates.

Of the above chelators, DTPA is a good binder of Indium-111, which a gamma emitter and thus a good diagnostic or imaging isotope. On the other hand, DOTA is a good binder for Yttrium-90, which is a beta-emitter, and thus is a good isotope for treatment purposes.

As far as it is known, all tumors have up regulated transferrin receptors, so that the present invention can be used of imaging tumors in the diagnosis, prognosis and follow-up of cancer patients; for the treatment/diagnosis of certain infectious diseases where either the disease vector or the infected cell manifest transferrin receptors; or for the identification and/or deletion of aggressive T-lymphocytes or B-lymphocytes in autoimmune diseases or in the elimination of the rejecting cells in patients with transplanted cells or organs.

In addition to the use of the complexes of the present invention as anti-tumor agents, the complexes can be used for the targeted delivery of cytotoxic drugs to activated lymphocytes responsible for the rejection of transplanted tissues and to transport high concentrations of radiosensitizers to cancer cells, and these uses, as well as the use of transcobalamin as a binding moiety that binds to a specific receptor on selected cells, are described in International Application No. PCT/US01/20444, of the present inventor, the disclosure of which is hereby incorporated by reference for such teachings therein. The use of conjugates for the treatment of parasitic infections is described in International Application No. PCT/US02/11893 of the present inventor, the disclosure of which is hereby incorporated by reference for the teachings of such treatment therein.

The targeted delivery of drugs to stressed cells, especially cells stressed as a result of a viral infection, is described in International Application PCT/US02/11892 of the present inventor, the disclosure of which is hereby incorporated by reference for such teachings therein. The conjugates of the present invention may be used in all of these treatments.

While the use of the materials of the present invention for the treatment of cancers is a preferred embodiment of the method of treatment aspects of the present invention, it will be clear that the present invention can broadly be used to identify and/or eliminate certain populations of cells, by using proteins that selectively bind to that population of cells, together with cell imaging and/or cell killing agents.

The methods of administration and the dosage of the materials of the present invention are similar to those used for doxorubicin-transferrin conjugates, as described in U.S. Pat. No. 5,108,987, of the present inventor, the disclosure of which is hereby incorporated by reference for the teaching of such methods of administration and dosage amounts therein.

The present invention has been augmented by reliance upon the teachings of the references cited herein below. The disclosure of these references is hereby incorporated by reference for the teachings referred to in this specification.

Validation of the Conjugates:

By using HPLC and/or polyacrylamide gel electrophoresis as described in (45), the homogeneity of metal-loaded transferrin-doxorubicin conjugates was determined. Similarly, by using spectrophotometry, the molecular ratio of doxorubicin-to-transferrin was determined (71). In addition, the ratio of doxorubicin-to-transferrin can be determined by using antibodies to doxorubicin deposited on the gold surface of a surface plasmon resonance (SPR) grating. The SPR anomaly moves in wavelength proportional to the mass of doxorubicin that binds to the anti-doxorubicin antibodies on the gold surface. Texas Instruments manufactures an SPR measurement system, the modifications of which have the required resolution and sensitivity for this application. An interesting aspect of this method is that it can be used for monitoring doxorubicin concentrations in the blood of cancer patients being treated with metal-loaded transferrin-doxorubicin conjugates. Experimental data indicate that a useful ratio of doxorubicin-to-transferrin is 2-to-1.

The ratio of metal-to-transferrin was determined by ultraviolet spectroscopy. In addition, the ratio of metal-to-transferrin is measured in a flow cell with two parallel metal plates. When an alternating current signal of appropriate frequency for the metal being quantified is applied to the plates, the impedance measured between the plates varies as a function of the amount of metal bound by the transferrin component of the transferrin-doxorubicin conjugates. Experimental data indicate that a useful ratio metal-to-transferrin is two atoms of metal per molecule of transferrin.

Conjugates additionally are validated for their ability to bind and kill specific cells. Binding studies are done with HL60 cells, K562 cells and normal peripheral blood lymphocytes by using fluorescence activated cell sorter analysis to determine if the conjugates bind to the cancer cells but not to normal cells. By using in vitro culture techniques as described in (45), cell killing studies were done with the same cancer cells and normal peripheral blood lymphocytes to validate that the conjugates kill cancer cells but not normal cells. These validation procedures also serve as quality controls for the conjugates.

In Vitro Studies of the Conjugates:

Each metal-loaded transferrin-doxorubicin conjugate was studied for its ability to kill drug-sensitive and drug-resistant K562 and HL60 cells. It should be noted that drug-resistant cells have significantly more transferrin receptors than drug-sensitive cells (148). Thus, the LD₅₀ for each experiment is compared to LD₅₀ values obtained by using drug-sensitive and drug-resistant cells cultured with non-metal-loaded transferrin-doxorubicin conjugates; metal-loaded transferrins that are not conjugated to doxorubicin; free metal, and free doxorubicin. By using cultures of multi-drug resistant human cancer cells as described in (106) it was found that non-metal-loaded transferrin-doxorubicin conjugates produced LD₅₀ values that were substantially less (i.e., often an order-of-magnitude less) than free doxorubicin against drug-resistant K562 and HL60 cells, and that metal-loaded transferrin-doxorubicin conjugates killed drug-resistant cells at even lower LD₅₀ values than non-metal-loaded transferrin-doxorubicin conjugates.

Animal Studies of the Conjugates:

Sprague-Dawley rats with chemically induced drug-resistant tumors have prolonged survival when they are treated with ruthenium (142), which complexes to the metal-binding sites of transferrin (129). Other studies have reported that nude mice bearing drug-resistant human tumors survive longer when treated with glutaraldehyde-prepared transferrin-doxorubicin conjugates than when treated with free doxorubicin (99), providing proof-of-principle that transferrin-doxorubicin conjugates kill drug-resistant human cancer cells in a mouse model. Similarly, the above results with ruthenium in drug-resistant tumor-bearing rats suggest that transferrin-metal complexes are effective against drug-resistant tumors.

Drug-sensitive and drug-resistant human cancer cells were studied in nude mice to test whether animals inoculated with a lethal dose of tumor cells and treated with metal-loaded transferrin-doxorubicin conjugates (measured as the amount of doxorubicin) survive significantly longer (i.e., p value equal to or less than 0.05) than animals inoculated with nothing, free doxorubicin, free metal, non-metal-loaded transferrin-doxorubicin, and metal-loaded transferrin. In these experiments, the null hypothesis is that metal-loaded transferrin-doxorubicin will not significantly prolong life as compared to non-metal-loaded transferrin-doxorubicin, metal-loaded transferrin, free metal or free doxorubicin, and the alternative hypothesis is that animals inoculated with metal-loaded transferrin-doxorubicin conjugates will survive significantly longer than animals inoculated with non-metal-loaded transferrin-doxorubicin metal-loaded transferrin, free metal or free doxorubicin. In addition, dose range-finding experiments are performed for each of the four metal-loaded transferrin-doxorubicin conjugates to determine maximal survival through a range of doxorubicin concentrations in metal-loaded transferrin-doxorubicin conjugates compared to animals in parallel experiments given non-metal-loaded transferrin-doxorubicin, metal-loaded-transferrin, free metal or free doxorubicin.

References

1. Faulk W P and Johnson P M. Immunological studies of human placentae.

Identification and distribution of proteins in mature chorionic villi. Clin Exp Immunol 1977; 27: 365-375.

2. Faulk W P, Johnson P M, Dorling J and Temple A. Non-specific factors of resistance in human placentae. Prot Biol Fluids 1976; 24: 139-142.

3. Johnson P M and Faulk W P. Immunological studies of human placentae: Identification and distribution of proteins in immature chorionic villi. Immunology 1978; 34: 1027-1035.

4. Faulk W P and Galbraith G M P. Trophoblast transferrin and transferrin receptors in the host-parasite relationship of human pregnancy. Proc R Soc Lond B 1979; 204: 83-97.

5. Hsi B L, Yeh C J G and Faulk W P. Human amniochorion: Tissue-specific markers, transferrin receptors and histocompatibility antigens. Placenta 1982; 3: 1-12.

6. Yeh C J G, Hsi B L and Faulk W P. Histocompatibility antigens, transferrin receptors and extra-embryonic markers of human amniotic epithelial cells in vitro. Placenta 1983; 4: 361-368.

7. Galbraith G M P, Galbraith R M and Faulk W P. Transferrin binding by human lymphoblastoid cell lines and other transformed cells. Cell Immunology 1980; 49: 215-222.

8. Faulk W P, Hsi B L and Stevens P J. Transferrin and transferrin receptors in carcinoma of the breast. Lancet 1980; ii: 390-392.

9. Yeh C J G, Taylor C and Faulk W P. Transferrin binding by peripheral blood mononuclear cells in human lymphomas, myelomas and leukemias. Vox Sanguinis 1984; 46: 217-223.

10. Faulk W P, Harats H and Berczi A. Transferrin receptor growth control in normal and abnormal cells. In: Oxidoreduction at the Plasma Membrane. Vol 1. (eds., F L Crane, J D Morre and H Low) CRC Press, Boca Raton, Fla., 1990; pp. 205-224.

11. Yang D C, Wang F, Elliott R L and Head J F. Expression of transferrin receptor and ferritin H-chain mRNA are associated with clinical and histopathological prognostic indicators in breast cancer. Anticancer Res 2001; 21: 541-549.

12. Barnett D, Wilson G A, Lawrence A C and Buckley G A. Transferrin receptor expression in the leukaemias and lymphoproliferative disorders. Clin Lab Haematol 1987; 9: 361-70.

13. Whitney J F, Clark J M, Griffin T W, Gautam S and Leslie K O. Transferrin receptor expression in nonsmall cell lung cancer. Histopathologic and clinical correlates. Cancer 1995; 76: 20-25.

14. Recht L, Torres C O, Smith T W, Raso V and Griffin T W. Transferrin receptor in normal and neoplastic brain tissue: implications for brain-tumor immunotherapy. J Neurosurg 1990; 72: 941-945.

15. Sciot R, Paterson A C, van Eyken P, Callea F, Kew M C and Desmet V J. Transferrin receptor expression in human hepatocellular carcinoma: an immunohistochemical study of 34 cases. Histopathol 1988; 12: 53-63.

16. Seymour G J, Walsh M D, Lavin M F, Strutton G and Gardiner R A. Transferrin receptor expression by human bladder transitional cell carcinomas. Urol Res 1987; 15: 341-344.

17. Lindholm M L, Lindberg L A, Vilja P, Puolakka V M, Nordling S, Schroder T and Schroder J. Expression of the human transferrin receptor in subrenal capsule assay in the mouse. J Surg Oncol 1988; 38: 57-62.

18. Hereiz H A and Bayoumi F A. Evaluation of diagnosis of ovarian malignancy using immunohistochemical technique. J Egyptian Public Hlth Assoc 1992; 67: 697-707.

19. Medeiros L J, Picker L J, Horning S J and Warnke R A. Transferrin receptor expression by non-Hodgkin's lymphomas. Correlation with morphologic grade and survival. Cancer 1988; 61: 1844-1851.

20. Soyer H P, Smolle J, Tome R and Kerl H. Transferrin receptor expression in normal skin and in various cutaneous tumors. J Cutaneous Pathol 1987; 14: 1-5.

21. Lesley J, Hyman R, Schulte R and Trotter J. Expression of transferrin receptor on murine hematopoietic progenitors. Cell Immunol 1984; 83: 14-25.

22. Testa U, Pelosi E and Peschle C. The transferrin receptor. Crit Rev Oncogen 1993; 4: 241-276.

23. Bothwell T A, Charlton R W, Cook J D and Finch C A. Iron Metabolism in Man, Blackwell Scientific, Oxford, 1979.

24. Ponka P and Lok C N. The transferrin receptor: role in health and disease. Int J Biochem Cell Biol 1999; 31: 1111-1137.

25. Hamilton T A, Gray P W and Adams D O. Expression of the transferrin receptor on murine peritoneal macrophages is modulated by in vitro treatment with interferon gamma. Cell Immunol 1984; 89: 478-488.

26. Byrd T F and Horowitz M A. Interferon gamma-activated human monocytes downregulate transferrin receptors and inhibits the intracellular multiplication of Legionella. pneumophila by limiting the availability of iron. J Clin Invest 1989; 83: 1457-1465.

27. Kronke M, Leonard W, Depper J M and Greene W C. Sequential expression of genes involved in human T lymphocyte growth and differentiation. J Exp Med 1985; 161: 1593-1598.

28. Galbraith R M and Galbraith G M. Expression of transferrin receptors on mitogen-stimulated human peripheral blood lymphocytes: relation to cellular activation and related metabolic events. Immunology 1983; 133: 703-710.

29. Neckers L M and Cossman J. Transferrin receptor induction in mitogen-stimulated human T lymphocytes is required for DNA synthesis and cell division and is regulated by interleukin 2. Proc Nat Acad Sci USA 1983; 80: 3494-3498.

30. Testa U, Kuhn L, Petrini M, Quaranta M T, Pelosi E and Peschle C. Differential regulation of iron regulatory element-binding protein(s) in cell extracts of activated lymphocytes versus monocytes-macrophages. J Biol Chem 1991; 266: 3925-3930.

31. Seiser C, Texieira S and Kuhn L C. Interleukin-2-dependent transcriptional and post-transcriptional regulation of transferrin receptor mRNA. J Biol Chem 1993; 268: 13,074-13,080.

32. Neckers L M, Yenokida G and James S P. The role of the transferrin receptor in human B lymphocyte activation. J Immunol 1984; 133: 2437-2441.

33. Neckers L M and Trepel J B. Transferrin receptor expression and the control of cell growth. Cancer Invest 1986; 4: 461470.

34. Yeh C J G, Papamichail M and Faulk W P. Loss of transferrin receptors following induced differentiation of HL-60 promyelocytic leukemia cells. Exper Cell Res 1982; 138: 429-431.

35. Barker K A and Newburger P E. Relationships between the cell cycle and the expression c-myc and transferrin receptor genes during induced myeloid differentiation. Exper Cell Res 1990; 186: 1-5.

36. Klausner R D, Rouault T A and Harford J B. Regulating the fate of mRNA: the control of cellular iron metabolism. Cell 1993; 72: 19-28.

37. Haile D J. Regulation of genes of iron metabolism by the iron-response proteins. Am J Med Sciences 1999; 318: 230-240.

38. Gatter K C, Brown G, Trowbridge I S, Woolston R E and Mason D Y. Transferrin receptors in human tissues: their distribution and possible clinical relevance. J Clin Pathol 1983; 36: 539-545.

39. Faulk W P and Hunt J S. Human placentae: view from an immunological bias. Am J Reprod Immunol 1990; 21: 108-113.

40. Broadwell R D, Baker-Caims B J, Friden P M, Oliver C and Villegas J C. Transcytosis of protein through the mammalian cerebral epithelium and endothelium. III. Receptor mediated transcytosis through the blood-brain barrier of blood-borne transferrin and antibody against transferrin receptor. Exp Neurol 1996; 142: 47-65.

41. Ponka P, Beaumont C and Richardson D R. Function and regulation of transferrin and ferritin. Seminars in Hematol 1998; 35: 35-54.

42. Sylvester S R and Griswold M D. The testicular iron shuttle: A “nurse” function of the Sartoli cells. J Androl 1994; 15: 381-385.

43. Yeh C J G and Faulk W P. Killing of human tumor cells in culture with adriamycin conjugates of human transferrin. Clin Immunol Immunopath 1984; 32: 1-11.

44. Yeh C J G, Taylor C G and Faulk W P. Targeting of cytotoxic drugs by transferrin receptors: Selective killing of acute myelogenous leukemia cells. Protides Biol Fluids 1984; 32: 441-444.

45. Berczi A, Barabas K, Sizensky J A and Faulk W P. Adriamycin conjugates of human transferrin bind transferrin receptors and kill K562 and HL60 cells. Arch Biochem Biophys 1993; 300: 356-363.

46. Lai B T, Gao J P and Lanka K W. Mechanism of action and spectrum of cell lines sensitive to a doxorubicin-transferrin conjugate. Cancer Chemother & Pharmacol 1998;41: 155-160.

47. Kratz F, Beyer U, Roth T, Tarasova N, Collery P, Lechenault F, Cazabat A, Schumacher P, Unger C and Falken U. Transferrin conjugates of doxorubicin: synthesis, characterization, cellular uptake, and in vitro efficacy. J Pharm Sciences 1998; 87: 338-346.

48. Tanaka T, Kaneo Y and Miyashita M. Synthesis of transferrin-mitomycin C conjugate as a receptor-mediated drug targeting system. Biol Pharm Bull 1996; 19: 774-777.

49. Sasaki K, Kohgo Y, Kato J, Kondo H and Niitsu Y. Intracellular metabolism and cytotoxicity of transferrin-neocarzinostatin conjugates of differing molar ratios. Jpn J Cancer Res 1993; 84: 191-196.

50. Laske D W, Youle R J and Oldfield E H. Tumor regression with regional distribution of the targeted toxin TF-CRM107 in patients with malignant brain tumors. Nature Med 1997; 3: 1362-1368.

51. Beyer U, Roth T, Schumacher P, Maier G, Unold A, Frahm A W, Fiebig H H, Unger C and Kratz F. Synthesis and in vitro efficacy of transferrin conjugates of the anticancer drug chlorambucil. J Med Chem 1998; 41: 2701-2708.

52. Bicamumpaka E and Page M. In vitro cytotoxicity of paclitaxel-transferrin conjugate on H69 cells. Oncol Reports 1998; 5: 1381-1383.

53. Lemieux P, Page M and Noel C. In vivo cytotoxicity and antineoplastic activity of a transferrin-daunorubicin conjugate. In Vivo 1992; 6: 621-627.

54. Guo M, Sun H, McArdle H J, Gambling L and Sadler P J. Ti(IV) uptake and release by human serum transferrin and recognition of Ti(IV)-transferrin by cancer cells: understanding the mechanism of action of the anticancer drug titanocene dichloride. Biochem 2000; 39: 10023-10033.

55. Shah D and Shen W C. Transcellular delivery of an insulin-transferrin conjugate in enterocyte-like Caco-2 cells. J Pharm Sciences 1996; 85: 1306-1311.

56. Drobyski W R, Ul-Haq R, Majewski D and Chitambar C R. Modulation of in vitro and in vivo T-cell responses by transferrin-gallium and gallium nitrate. Blood 1996; 88: 3056-3064.

57. Hoshino T, Misaki M, Yamamoto M, Shimizu H, Ogawa Y and Toguchi H. In vitro cytotoxicities and in vivo distribution of transferrin-platinum(II) complex. J Pharm Sciences 1995; 84: 216-221.

58. Ippoliti R, Ginobbi P, Lendaro E, D'Agostino I, Ombres D, Benedetti P A, Brunori M and Citro G. The effect of monensin and chloroquine on the endocytosis and toxicity of chioneric toxins. Cell Mol Life Sci 1998; 54: 866-875.

59. Kratz F, Hartmann F, Keppler B and Messor L. The binding properties of two antitumor ruthenium(III) complexes to apotransferrin. J Biol Chem 1994; 269: 2581-2588.

60. Park E, Starzyk R M, McGrath J P, Lee T, George J, Schutz A J, Lynch P and Putney S D. Production and characterization of fusion proteins containing transferrin and nerve growth factor. J Drug Targeting 1998; 6: 53-64.

61. Ali S A, Joao H C, Hammerschmid F, Eder J and Steinkasserer A. Transferrin Trojan Horses as a rational approach for biological delivery of therapeutic peptide domains. J Biol Chem 1999; 274: 24066-24073.

62. Peters K and Richards F M. Chemical cross-linking: reagents and problems in studies of membrane structure. Annu Rev Biochem 1977; 46: 523-551.

63. Rhodes J. Evidence for an intercellular covalent reaction essential in antigen-specific T cell activation. J Immunol 1989; 143: 1482-1489.

64. Greenfield R S, Kaneko T, Daues A, Edson M A, Fitzgerald K A, Olech L J, Grattan J A, Spitalny G L and Braslawsky G R. Evaluation in vitro of adriamycin immunoconjugates synthesized using an acid-sensitive hydrazone bond. Cancer Res 1990; 50: 6600-6607.

65. Braslawsky G R, Edson M A, Pearce W, Kaneko T and Greenfield R S. Antitumor activity of adriamycin (hydrazone-linked) immunoconjugates compared with free adriamycin and specificity of tumor cell killing. Cancer Res 1990; 50: 6608-6614.

66. O'Keefe D O and Draper R K. Characterization of a transferrin-diphtheria toxin conjugate. J Biol Chem 1985; 260: 932-937.

67. Neidle S, Pearl L H and Skelly J V. DNA structure and perturbation by drug binding. Biochem J 1987; 243: 1-13.

68. Tritton T R. Cell surface actions of adriamycin. Pharmacol & Therapeutics 1991: 49: 293-309.

69. Maestre N, Tritton T R, Laurent G and Jaffrezou J P. Cell surface-directed interaction of anthracyclines leads to cytotoxicity and nuclear factor kappaB activation but not apoptosis signaling. Cancer Res 2001; 61: 2558-2561.

70. Fong W F, Lam W, Yang M and Wong JT-F. Partial synergism between dextran-conjugated doxorubicin and cancer drugs on the killing of multidrug resistant KB-V1 cells. Anticancer Res 1996; 16: 3773-3778.

71. Barabas K, Sizensky J A and Faulk W P. Transferrin conjugates of adriamycin are cytotoxic without intercalating nuclear DNA. J Biol Chem 1992; 267: 9437-9442.

72. Faulk W P, Barabas K, Sun I L and Crane F L. Transferrin-adriamycin conjugates which inhibit tumor cell proliferation without interaction with DNA inhibit plasma membrane oxidoreductase and proton release in K562 cells. Biochem Int 1991; 25: 815-822.

73. Berczi A, Ruthner M, Szuts V, Fritzer M, Schweinzer E and Goldenberg H. Influence of conjugation of doxorubicin to transferrin on the iron uptake by K562 cells via receptor-mediated endocytosis. Euro J Biochem 1993; 213: 427436.

74. Barabas K, Sizensky J and Faulk W P. Evidence in support of the plasma membrane as the target for transferrin-adriamycin conjugates in K562 cells. Am J Reprod Immunol 1991; 25: 120-124.

75. Szuts V, Berczi A, Schweinzer E and Goldenberg H. Binding of doxorubicin-conjugated transferrin to U937 cells. J Receptor Res 1993; 13: 1041-1054.

76. Ruthner M, Berczi A and Goldenberg H. Interaction of a doxorubicin-transferrin conjugate with isolated transferrin receptors. Life Sci 1994; 54: 35-40

77. Sainte-Marie J, Lafont V, Pecheur E I, Fa\grave{v}ero J, Philippot J R and Bienvenue A. Transferrin receptor functions as a signal-transduction molecule for its own recycling via increases in the internal Ca++ concentration. Euro J-Biochem 1997; 250: 689-697.

78. Klausner R D, vanReuswoude J, Ashwell G, Kempf C, Schechter A N, Dean A and Bridges K. Receptor-mediated endocytosis of transferrin in K562 cells. J Biol Chem 1983; 258: 47154724.

79. Richardson D R and Ponka P. The molecular mechanisms of a metabolism and transport of iron in normal and neoplastic cells. Biochim Biophy Acta 1997; 1331: 1-40.

80. Baker M A and Lawen A. Plasma membrane NADH-oxidase system: a critical review of the structural and functional data. Antioxidants & Redox Signaling 2000; 2: 197-212.

81. Sun I L, Navas P, Crane F L, Morre D J and Low H. NADH-diferric transferrin reductase in liver plasma membranes. J Biol Chem 1987; 262: 15915-15921.

82. Sun I L, Navas P, Crane F L, Morre D J and Low H. Diferric transferrin reductase in the plasma membrane is inhibited by adriamycin. Biochem Int 1987; 14: 119-127.

83. Faulk W P, Harats H, McIntyre J A, Berczi A, Sun I L and Crane F L. Recent advances in cancer research: Drug targeting without the use of monoclonal antibodies. Am J Reprod Immunol 1989; 21: 151-154.

84. Morre D J, Kim C, Paulik M, Morre D M and Faulk W P. Is the drug-response NADH-oxidase of the cancer cell plasma membrane a molecular target for adriamycin? Bioenerg Biomembr 1997; 29: 269-280.

85. Sun I L, Sun E E, Crane F L, Morre D J and Faulk W P. Inhibition of transplasma membrane electron transport by transferrin-adriamycin conjugates. Biochim Biophy Acta 1992; 1105: 84-88.

86. Crane F L, Low H, Sun I L, Morre D J and Faulk W P. Interaction between oxidoreductase, transferrin receptor and channels in the plasma membrane. In: Growth Factors from Genes to Clinical Applications (eds, V R Sara, K Hall and H Low) Raven Press, New York, 1990; pp. 228-239.

87. Hileti D, Panayiotidis P and Hoffbrand V. Iron chelators induce apoptosis in proliferating cells., Brit J Haematol 1995; 89: 181-187.

88. Leardi A, Caraglia M, Selleri C, Pepe S, Pizzi C, Notaro R, Fabbrocini A, De Lorenzo S, Musico M, Abbruzzese A, Bianco A and Tagliaferri P. Desferioxamine increases iron depletion and apoptosis induced by ara-C of human myeloid leukemic cells. Brit J Haematol 1998; 102: 746-752.

89. Barabas K, Miller S J and Faulk W P. Regulation of transferrin receptor mRNA stability in drug-sensitive and drug-resistant cancer cells. To be submitted for publication, 2003.

90. Hentze M W and Kuhn L C. Molecular control of vertebrate iron-metabolism: mRNA-based regulatory circuits operated by iron, nitric oxide and oxidative stress. Proc Natl Acad Sci USA 1996; 93: 8175-8182.

91. Pantapoulos K and Hentze M W. Rapid responses to oxidative stress mediated by iron regulatory protein. EMBO J 1995; 14: 2917-1924.

92. Wardrop S L, Watts R N and Richardson D R. Nitrogen monoxide activates iron regulatory protein 1 RNA-binding activity by two possible mechanisms: effect on the 4Fe-4S cluster and iron mobilization from cells. Biochemistry 2000; 39: 2748-2758.

93. Eisenstein R S. Iron regulatory proteins and the molecular control of mammalian iron metabolism. Annu Rev Nutr 2000; 20: 627-662.

94. Richardson D R, Naumannova V, Nagy E and Ponka P. The effect of redox-related species of nitrogen monoxide on transferrin and iron uptake and cellular proliferation of erythroleukemia (K562) cells. Blood 1995; 86: 3211-3219.

95. Kim S and Ponka P. Effects of interferon-gamma and lipopolysaccharide on macrophage iron metabolism are mediated by nitric oxide-induced degradation of iron regulatory protein 2. J Biol Chem 2000; 275: 6220-6226.

96. Nestel F P, Green R N, Kickian K, Ponka P and Lapp W S. Activation of macrophage cytostatic effector mechanisms during acute graft-versus-host disease: release of intracellular iron and nitric oxide-mediated cytostasis. Blood 2000; 96: 1836-1843.

97. Kim S and Ponka P. Control of transferrin receptor expression via nitric oxide-mediated modulation of iron-regulatory protein 2. J Biol Chem 1999; 274: 33035-33042.

98. Laske D W, Ilercil O, Akbasak A, Youle R J and Oldfield E H. Efficacy of direct intratumoral therapy with targeted protein toxins for solid human gliomas in nude mice. J Neurosurg 1994; 80: 520-526.

99. Singh M, Atwal H and Micetich R. Transferrin directed delivery of adriamycin to human cells. Anticancer Res 1998; 18(3A): 1423-1427.

100. Sato Y, Yamauchi N, Takahashi M, Sasaki K, Fukaura J, Neda H, Fujii S, Hirayma M, Itoh Y, Koshita Y, Kogawa K, Kato J, Sakamaki S and Niitsu Y. In vivo gene delivery to tumor cells by transferrin-streptavidin-DNA conjugate. FASEB Journal 2000; 14: 2108-2118.

101. Oldfield E H and Youle R J. Immunotoxins for brain tumor therapy. Cur Top Microbiol Immunol 1998; 234: 97-114.

102. Kohgo Y, Kato J, Sasaki K and Kondo H. Targeting chemotherapy with transferrin-neocarzinostatin. Japanese J Cancer Chemotherapy 1988; 15: 1072-1076.

103. Faulk W P, Taylor C G, Yeh G and McIntyre J A. Preliminary clinical study of transferrin-adriamycin conjugate for drug delivery to acute leukemia patients. Mol Biother 1990; 2: 57-60.

104. Laske D W, Morrison P F, Lieberman D M, Carthesy M E, Reynolds J C, Stewart-Henney P A, Koong S S, Cummins A, Paik C H and Oldfield E H. Chronic interstitial infusion of protein to primate brain: determination of drug distribution and clearance with single-photon emission computerized tomography imaging. J Neurosurg 1997; 87: 586-594.

105. Marbeuf-Gueye C, Ettori D, Priebe W, Kozlowski H and Garnier-Suillerot A. Correlation between the kinetics of anthracycline uptake and the resistance factor in cancer cells expressing the multidrug resistance protein or the P-glycoprotein. Biochem Biophy Acta 1999; 1450: 374-384.

106. Fritzer M, Barabas K, Szuts V, Berczi A, Szekeres T, Faulk W P and Goldenberg H. Cytotoxicity of a transferrin-adriamycin conjugate to anthracylcine resistant cells. Int J Cancer 1992; 52: 619-623.

107. Hatano T, Ohkawa K and Matsuda M. Cytotoxic effect of the protein-doxorubicin. conjugates on the multidrug-resistant human myelogenous leukemia cell line, K562, in vitro. Tumor Biology 1993; 14: 288-294.

108. Lemieux P and Page M. Sensitivity of multidrug-resistant MCF-7 cells to a transferrin-doxorubicin conjugate. Anticancer Res 1994; 14(2A): 397-403.

109. Fritzer M, Szekeres T, Szuts V, Jraayam H N and Goldenberg H. Cytotoxic effects of a doxorubicin-transferrin conjugate in multidrug-resistant KB cells. Biochem Pharm 1996; 51: 489-493.

110. Wang F, Jiang X, Yang D C, Elliot R L and Head J F. Doxorubicin-gallium-transferrin conjugate overcomes multidrug resistance: evidence for drug accumulation in the nucleus of drug resistant MCF-7/ADR cells. Anticancer Res 2000; 20: 799-808.

111. Soma C E, Dubernet C and Barratt G. Ability of doxorubicin-loaded nanoparticles to overcome multidrug resistance of tumor cells after their capture by macrophages. Pharm Res 1999; 16: 1710-1716.

112. Mazel M, Clair P, Rousselle C, Vidal P, Scherrmann J-M, Mathieu D and Temsamani J. Doxorubicin-peptide conjugates overcome multidrug resistance. Anti-Cancer Drugs 2001; 12: 107-116.

113. Anderson B F, Baker H M, Norris G E, Rumball S V and Baker E N. Apolactoferrin structure demonstrates ligand-induced conformational change in transferrins. Nature 1990; 344: 784-787.

114. Baker E N. Structure and reactivity of transferrin. Adv Inorg Chem 1994; 41: 389-463.

115. Harris W R. Equilibrium constants for the complexation of metal ions by serum transferrin. Adv Exp Med & Biol 1989; 249: 67-93.

116. Li H, Sadler P J and Sun H. Unexpectedly strong binding of a large metal ion (Bi³⁺) to human serum transferrin. J Biol Chem 1996; 271: 9483-9489.

117. Battistuzzi G, Calzolai L, Messori L and Sola M. Metal-induced conformational heterogeneity of transferrins: a spectroscopic study of indium (III) and other metals (III)-substituted transferrins. Biochem Biophys Res Com 1995; 206: 161-170.

118. Kubal G, Mason A B, Patl S U, Sadler P J and Woodworth R C. Oxolate- and Ga³⁺-induced structural changes in human transferrin and its recombinant N-lobe. ¹H NMR of preferential C-lobe Ga³⁺ binding. Biochem 1993; 32: 3387-3395.

119. Grossman J G, Neu M, Evans R W, Lindley P F, Appel H and Hasnain S S. Metal-induced conformational changes in transferrins. J Mol Biol 1993; 229: 585-590.

120. Sun H, Li H, Mason A B, Woodworth R C and Sadler P J. N-lobe versus C-lobe complexation of bismuth by human transferrin. Biochem J 1999; 337: 105-111.

121. Dobson C B, Graham J and Itzhaki R F. Mechanism of uptake of gallium by human neuroblastoma cells and effects of gallium and aluminum on cell growth, lysosomal protease, and choline acetyl transferase activity. Exp Neurol 1998; 153: 342-350.

122. Abreo K, Jangula J, Jain S K, Sella M and Glass J. Aluminum uptake and toxicity in cultured mouse hepatocytes. J Am Soc Nephrol 1991; 1: 1299-1304.

123. Sun H, Li H, Mason A B, Woodworth R C and Sadler P J. Competitive binding of bismuth to transferrin and albumin in aqueous solution and in blood plasma. J Biol Chem 2001; 276: 8829-8835.

124. Gallori E, Vettori C, Alessio E, Vilchez F G, Vilaplana R, Orioli P, Casini A and Messori L. DNA as a possible target for antitumor ruthenium complexes. Arch Biochem Biophy 2000; 376: 156-162.

125. Ward S G and Taylor R C. In, Metal-Based Anti-Tumor Drugs (Gielen M F, Ed) 1988, pp 1-54, Fruend Publishing House Ltd., London.

126. Kubal G and Sadler P J. Sequential binding of aluminum (3+) to the C- and N-lobe of human serum transferrin detected by ¹H NMR spectroscopy. J Am Chem Soc 1992; 114: 1117-1118.

127. Kubal G, Mason A B, Sadler P J, Tucker A and Woodworth R C. Uptake of Al³⁺ into the N-lobe of human serum transferrin. Biochem J 1992; 285: 711-714.

128. Van Rensburg S J, Carstens M E, Potocnik F C V and Taljaard J J F. The effect of iron and aluminum on transferrin and other serum proteins as revealed by isoelectric focusing gel electrophoresis. Annals NY Acad Sci 2000; 903: 150-155.

129. Kratz F, Hartmann M, Keppler B and Messori L. The binding properties of two antitumor ruthenium (III) complexes to apotransferrin. J Biol Chem 1994; 269: 2581-2588.

130. Guo M, Sun H, McArdle J H, Gambling L and Sadler P J. Ti^{IV} uptake and release by human serum transferrin and recognition of Ti^{IV}-transferrin by cancer cells: understanding the mechanism of action of the anticancer drug titanocene dichloride. Biochem 2000; 39: 10023-10033.

131. Roskams A J and Cosmor J R. Aluminum access to the brain: a role for transferrin and its receptor. Proc Natl Acad Sci USA 1990; 87: 9024-9027.

132. Knorr G M and Chitamber C R. Gallium-pyridoxal isonicotinoyl hydrazone (Ga—PIH), a novel cytotoxic gallium complex. A comparative study with gallium nitrate. Anticancer Res 1998; 18 (3A): 1733-1737.

133. Kasai K, Hori M T and Goodman W G. Transferrin enhances the antiproliferative effect of aluminum on osteoblast-like cells. Am J Physiol 1991; 260 (4Pt1): E537-543.

134. McGregor S J, Naves M L, Birly A K, Russell N H, Halls D, Junor B J and Brock J H. Interaction of aluminum and gallium with human lymphocytes: the role of transferrin. Biochim Biophys Acta 1991; 1095: 196-200.

135. Abreo K and Glass J. Cellular, biochemical, and molecular mechanisms of aluminium toxicity. Nephrol Dial Transplant 1993; 8 Suppl 1: 5-11.

136. Kratz F, Mulinacci N, Messori L, Bertini I and Keppler B K. In, Metal Ions in Biology and Medicine, Vol. 2, pp. 69-74, John Libbey Limited Eurotext, Paris.

137. WiSniewski M Z, Wietrzyk J and Opolski A. Novel Ru(III), Rh(III), Pd(II) and Pt(II) complexes with ligands incorporating azole and pyrimidine rings. I. Antiproliferative activity in vitro. Arch Immunolog Therap Exper 2000; 48: 51-55.

138. Frasca D R, Gehrig L E and Clarke M J. Cellular effects of transferrin coordinated to. J Inorg Biochem 2001; 83: 139-149.

139. Whelan H R, Williams M B, Bijic D M, Flores R E, Schmidt M H, McAuliffe T L and Chitambar C R. Gallium nitrate delays the progression of microscopic disease in a human medulloblastoma murine model. Ped Neurol 1994; 11: 44-46.

140. Ganot P O. Metabolism and possible health effects of aluminum. Envir Hlth Perspect 1986; 65: 363-441.

141. Keppler B K, Berger M R, and Heim M E. New tumor-inhibiting metal complexes. Cancer Treat Rev 1990; 17: 261-277.

142. Seelig M H, Berger M R and Keppler B K. Antineoplastic activity of three ruthenium derivatives against chemically induced colorectal carcinoma in rats. J Cancer Res Clin Oncol 1992; 188: 195-200.

143. Webster L K, Olver I N, Stokes K H, Sephton R G, Hillcoat B L and Bishop J F. A pharmacokinetic and phase II study of gallium nitrate in patients with non-small cell lung cancer. Cancer Chemother & Pharmacol 2000; 45: 55-58.

144. Brechbiel M W. Chelated metal ions for therapeutic and diagnostic applications. Exper Biol & Med 2001; 226: 627-628.

145. Veronese I, Giussani A, Cantono M C, de Bartolo D, Roth P and Werner E. Kinetics of systemic ruthenium in human blood using a stable tracer. J Radiol Protect 2001; 21: 31-38.

146. Crul M, van den Bongard H J, Tibben M M, van Tellingen O, Sava G, Schellens J H and Beijnen J H. Validated method for the determination of the novel organo-ruthenium anticancer drug NAMI-A in human biological fluids by Zeeman atomic absorption spectrometry. Fresenius J Anal Chem 2001; 369: 442-445.

147. Howard J B and Rees D C. Perspectives on non-heme iron protein chemistry. Adv Protein Chem 1991; 42: 199-280.

148. Barabas K and Faulk W P. Transferrin receptors associate with drug resistance in cancer cells. Biochem Biophys Res Corn 1993; 197: 702-708.

149. Luttropp C A, Jackson J A, Jones B J, Sohn M H, Lynch R E and Morton K A. Uptake of Gallium-67 in transfected cells on tumors absent or enriched in the transferrin receptors. J Nucl Med 1998; 39: 1405-1411.

150. Pannccio M, Zalcberg J R, Thompson C H, Leyden J M, Sullivan J R, Lichtenstein M and McKenzie I F. Heterogeneity of the human transferrin receptor and use of anti-transferrin receptor antibodies to detect tumors in vivo. Immunol & Cell Biol 1987; 65: 461472.

151. Farley J, Loup D, Nelson M, Miller M J, Taylor R and Gray K. Transferrin in normal and neoplastic endocervical tissues: distribution and receptor expression. Analyst & Quant Cytol & Histol 1998; 20: 238-249.

152. Sausville E A and Feigal E. Evolving approaches to cancer drug discovery and development at the National Cancer Institute, USA. Annals Oncol 1999; 10: 1287-1291.

153. Surolia N and Misquith S. Cell surface directed targeting of toxin to human malaria parasite. FEBS Lett 1996; 396:57-61

154. Ohno H, Aguilar R C, Fournier M-C, Hennecke S, Cosson P and Boifacirio J S. Interaction of endocytic signals from the HIV-1 envelope glycoprotein complex with members of the adaptor medium chain family. Virology 1997; 238: 305-315.

155. Woodward J E, Bayer A L and Baliga P. Enhanced allograft survival via simultaneous blockade of transferrin receptor and interleukin-2-receptor. Transplantation 1999; 68: 1369-1376.

156. Som P, Oster Z H, Matsui K, Guglielmi G, Persson B R, Pellettieri M L, Srivastrava S C, Richards P, Atkins H L and Brill A B. 97Ru-transferrin uptake in tumor and abscess. Eur J Nucl Med 1983; 8: 491-494.

157. Lambert J R. Pharmacology of bismuth-containing compounds. Rev Inf Dis 1991; 13(Suppl 8): S691-S695.

158. Pariente J L, Bordenave L, Bareille R, Ohayon-Courtes C, Baquey C and LeGuillou M. In vitro cytocompatibility of radio-opacifiers used in ureteral endoprosthesis. Biomaterials 1999; 20: 523-527.

159. Krari N, Mauras Y and Allain P. Enhancement of bismuth toxicity by L-cysteine. Res Corn Mol Pathol & Pharmacol 1995; 89: 357-364.

160. Stoltenberg M, Schionning S and Danscher G. Retrograde axonal transport of bismuth: an autometrallographic study. Acta Neuropathol 2001; 101: 123-128.

1. A material comprising a protein that selectively binds to selected cells, the protein carrying a plurality of different cell-affecting entities.

2. The material of claim 1, wherein the selected cells are tumor cells.

3. The material of claim 2, wherein the protein is transferrin, and the tumor cells have up-regulated transferrin binding sites.

4. The material of claim 1, wherein the entities are selected from the group consisting of drugs, metals, radioisotopes, imaging aids and mixtures thereof.

5. The material of claim 1, wherein at least one of the entities is an anti-tumor agent.

6. The material of claim 5, wherein the anti-tumor agent is doxorubicin.

7. The material of claim 1, wherein at least one of the entities is a metal which kills tumor cells.

8. The material of claim 7, wherein the metal is bismuth.

9. The material of claim 1, wherein at least one of the entities is an imaging metal.

10. The material of claim 9, wherein the imaging metal is gallium.

11. The material of claim 6, wherein at least a second of the entities is a metal.

12. The material of claim 11, wherein the metal kills tumor cells.

13. The material of claim 1 1, wherein the metal images the selected cells.

14. The material of claim 1, wherein at least one of the entities is bound to the protein through a linker.

15. The material of claim 1, wherein at least one of the entities is bound to the protein through a chelator.

16. A method of treating and/or imaging diseased cells, and method comprising contacting the diseased cells with a material of claim 1 wherein the protein selectively binds to the diseased cells.

17. The method of claim 16, wherein the method is conducted in vivo in a patient in need of such treatment.

18. The method of claim 17, wherein the diseased cells are tumor cells.

19. The method of claim 17, wherein at least one of the entities is active against the disease.

20. The method of claim 17, wherein at least one of the entities images the diseased cells.

21. A method of treating a disease in a patient having need of such treatment, said treatment comprising administering an anti-disease effective amount of a material of claim 1 to the patient, wherein the selected cells are diseased, and at least one of the entities is active against the disease.

22. The method of claim 21, wherein at least one of the entities is doxorubicin or cisplatin.

23. A method of imaging selected cells in a patient, comprising administering to the patient an imaging-effective amount of a material of claim 1, wherein at least one of the entities is an imaging agent.
